(19) United States
(12) Patent Application Publication
(10) Pub. No.: US 2008/0132817 A1
(43) Pub. Date: Jun. 5, 2008

(54) Brace for use with an external fixation device

(76) Inventor: George R. Vito, Macon, GA (US)

Correspondence Address: Stephen B. Salai, Esq.;Harter, Secrest & Emery LLP, 1600 Bausch & Lomb Place, Rochester, NY, 14604-2711, US

(21) Appl. No.: 11/566,165

(22) Filed: Dec. 1, 2006

(51) Int. Cl. A61F 5/01

(52) U.S. Cl. 602/23

(57) ABSTRACT

An external fixation device is provided for substantially immobilizing a patient's foot relative to the patient's leg, after a conventional procedure in which wires are disposed through the foot and opposite ends of the pins are secured to a foot frame arranged proximate the patient's foot. The fixation device includes a pair of braces, a cushion, and a fastener. The braces are arranged on opposite sides of the patient's leg, and each has a plurality of attachment points for selectively receiving a first end of a stabilizing rod. The cushion is disposed between each of the braces and the patient's leg. The fastener secures the braces and the cushion relative to the patient's leg. The stabilizing rod is adjustably secured proximate a second end to the foot frame.

### BACKGROUND OF THE INVENTION

1. Field of the Invention

The present invention relates generally to therapeutic devices for use with known bone fixation procedures. More specifically, the invention relates to a therapeutic brace for immobilizing a foot and ankle with respect to a leg.

2. Description of Related Art

Procedures are well known for correcting bone fractures and deformities, and in particular fractures and deformities to the foot and ankle. At least one of those procedures was introduced by Dr. Ilizarov. As applied to a foot deformity, the Ilizarov procedure involves inserting wires percutaneously through bones or bone segments of the foot. Opposite ends of the wire are then secured to a frame disposed about all or a portion of the foot.

As is also generally understood, once the bones in the foot an/or ankle are appropriately aligned using the wires, it is desirable to prohibit movement of the foot with respect to the leg. To this end, the Ilizarov procedure includes the insertion of wires percutaneously through the tibia and/or the fibula. These wires are used to secure rings disposed around the patient's leg, and rods or bolts are used to secure the frame disposed around the foot with respect to the rings disposed around the lower leg.

Of course, the insertion of wires through one's skin and bones is not without some medical risks. For example, skin irritation around the site at which the wire is inserted through the leg is not uncommon. Infection also may occur at these sites. Furthermore, if a wire breaks, the wire may need to be replaced. More serious risks, such as neurological damage resulting from improper drilling for insertion of the wires may result in irreversible problems. Amputation may also be necessary in extreme cases. Moreover, the Ilizarov procedure also creates several problems with respect to post operative care and recovery.

U.S. Pat. No. 6,694,663 describes a combination bone fixation/immobilization boot that stabilizes a patient's foot and ankle without the use of wires through the lower leg. However, the boot of the '633 patent also has several drawbacks. For example, the boot is complex and costly to manufacture. Furthermore, the boot is not adaptable to conventional foot plates, and is not easily removable for purposes of inspecting/cleaning the patient's lower leg.

Accordingly, there is a need in the art for an improved method of stabilizing a foot and an ankle with respect to a leg. Moreover, there is need in the art for an apparatus for stabilizing a foot and an ankle with respect to a leg that does not require the insertion of wires through the leg. In addition, there is a need in the art for an apparatus for immobilizing a foot and ankle with respect to a leg that is simplistic and cheaply manufactured, and readily removable for inspection and cleaning of the leg.

### SUMMARY OF THE INVENTION

The present invention remedies the foregoing problems in the art by providing an external fixation device.

In a first aspect of the invention, an external fixation device is provided for substantially immobilizing a patient's foot relative to the patient's leg, after a conventional procedure in which wires are disposed through the foot and opposite ends of the pins are secured to a foot frame arranged proximate the patient's foot. The fixation device includes a pair of braces a cushion, and a fastener. The braces are arranged on opposite sides of the patient's leg, and each has a plurality of attachment points for selectively receiving a first end of a stabilizing rod. The cushion is disposed between each of the braces and the patient's leg. The fastener secures the braces and the cushion relative to the patient's leg. The stabilizing rod is adjustably secured proximate a second end to the foot frame.

In a further aspect of the present invention, an external fixation apparatus for use in immobilizing a foot with respect to an ankle includes a plurality of pins for insertion through a patient's foot, a foot frame, a plurality of stabilizing rods, a selectively attachable sleeve, a pair of braces, a fastener, and a foot pad. The foot frame surrounds at least a portion of the patient's foot, and includes a plurality of attachment points for attachment of distal ends of the pins. The plurality of stabilizing rods has a first end and a second end, the first end being selectively attachable to the foot frame. The selectively attachable sleeve is for placement on the patient's leg. The pair of braces are disposed along a portion of the patient's leg, on the sleeve, each brace having a plurality of apertures therethrough providing attachment points for selective attachment of the second end of the stabilizing rods. The fastener maintains the braces relative to the sleeve. The foot pad has a tread formed on a bottom thereof for selective attachment relative to the sole of the patient's foot.

In yet another aspect of the invention, an external fixation device is provided for use with a foot reconstruction technique, the technique comprising inserting pins through a patient's foot and securing the pins to a foot frame at least partially surrounding the patient's foot. The fixation device includes a sleeve, a pair of braces, and at least one removable strap The sleeve is open on opposite ends and is selectively attachable over a portion of the patient's leg. The braces are disposed on the sleeve on opposite sides of the patient's leg, generally along the patient's leg. The at least one removable strap is used for securing at least one of the sleeve and the braces relative to the patient's leg. A plurality of rods are secured proximate opposite ends to the foot frame and one of the braces to substantially inhibit movement of the foot frame relative to the braces, and thus immobilize the patient's foot relative to the patient's leg.

An understanding of these and other features of the present invention may be had with reference to the attached figures and following description, in which the present invention is illustrated and described

### BRIEF DESCRIPTION OF THE SEVERAL VIEWS OF THE DRAWINGS

FIG. 1 is a perspective view of a preferred embodiment of the immobilization apparatus according to the present invention.

FIGS. 2A, 2B, and 2C are perspective and side views of a preferred brace according to the present invention

FIG. 3 is a perspective view of the immobilization apparatus according to the present invention, as applied to a patient.

### DETAILED DESCRIPTION OF THE PREFERRED EMBODIMENTS

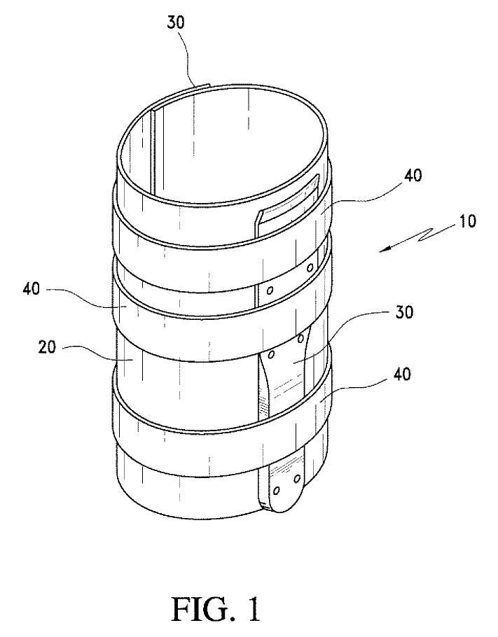

Preferred embodiments of the present invention will be described with reference to the Figures. FIGS. 1 through 3 show a first preferred embodiment of the invention. As illustrated in FIG. 1, an immobilization apparatus 10 according to the present invention includes a sleeve 20, a pair of braces 30, and a plurality of straps 40.

The sleeve 20 preferably is selectively attachable to a patient's leg. The sleeve 20 may be an elastic sheath to be slipped over the foot, but preferably is a unitary piece shaped substantially rectangularly. This rectangular piece is rolled or wrapped around the leg and subsequently secured firmly about the leg. Preferably, the sleeve utilizes hook and loop fasteners as a means for fastening the sleeve about the legs. More specifically, an outer portion of the sleeve may comprise the loop portion of the hook and loop fastener while a strip of hooks are formed on a facing part of the sleeve, in this manner, the sheath is adjustable to fit numerous patients' legs.

Of course, other attachment mechanisms may be used to attach the sheath about the leg. For example, clips, clasps, snaps, buttons, hooks, and the like all are within the knowledge of one of ordinary skill in the art as methods for fastening the sheath about the patient's leg.

The sleeve 20 preferably is a padded sleeve and may be made of any number of materials. The preferred sleeve is constructed of a valour or similar material that comprises the loop portion of a hook and loop fastening system. Of course, other materials, including textiles, plastics, and the like may also be used. The sleeve also may comprise a multi-layer construction, for example, a padded layer disposed between inner and outer layers, to further protect the patient's leg from the braces 30.

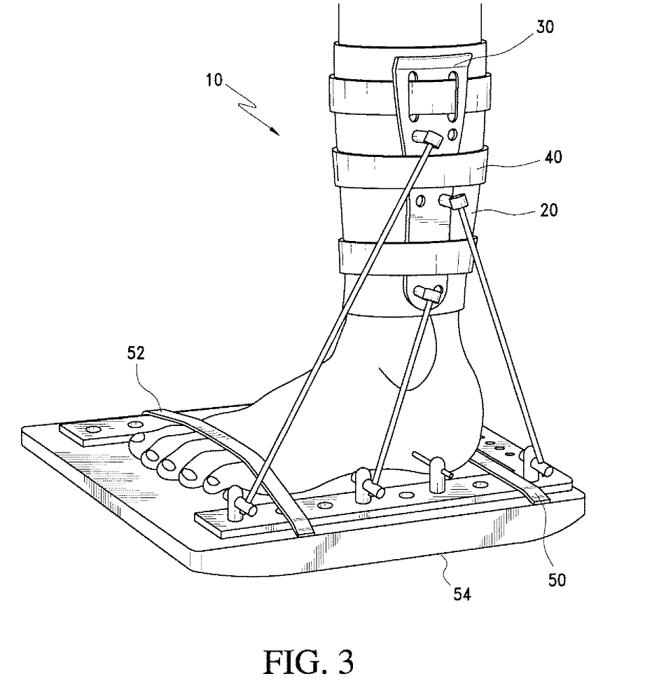

As illustrated best in FIGS. 3a and 3b, the brace 30 according to the invention includes a first portion 32 and a second portion 34. The first and second portions 32, 34 preferably are flexible relative to each other. This flexibility may be achieved by a flexure line between the first and second portions 32,34 or by virtue of the first potion 32 being inherently more flexible than the second portion 34, or vice versa. Preferably, two braces 30 are provided and are worn over the sheath 20 on opposite sides of the leg, in a manner similar to stirrups. Each of the braces 30 preferably is made of a substantially rigid plastic. Of course, any number of materials including plastics, ceramics, metals, and wood may be used for the braces.

In the preferred embodiment, the first portion 32 is an upper portion arranged higher on the leg than the second, lower portion 34. Moreover, the surface of the upper portion 32 that contacts the patient's leg preferably is generally convex to substantially fit the contours of the patient's leg. Of course, the second portion 34 may also/alternatively be contoured to control to the patient's leg. To further ensure an adequate fit of the braces, and as described above, the first portion 32 preferably is flexible with respect to the second portion 34 of the brace 30 such that when the brace is applied to the leg, the upper portion preferably is allowed to flex outwardly, away from the leg, for example to accommodate the widening of the leg at distances from the ankle.

As also illustrated, the first portion 32 preferably has a relatively larger surface area for contacting an adjacent portion of the patient's leg than the second portion 34. Specifically, the inventor has found that the increased surface area of the first portion 32 helps to maintain a substantially similar ratio of brace to leg at all distances from the ankle. This similar ratio provides for equal distribution across the leg of forces applied to the braces.

The brace 30 may further include fastening means 38 for assisting in securing the brace to the sheath 20. In the preferred embodiment, a section, e.g., a strip or tab, of hooks comprising half of a hook and loop fastener system is disposed on the side of the brace 30 for contacting the patient's leg. This section of hooks preferably cooperates with the leg sheath, which as described above, is made of a material acting as the loop portion of a hook and loop fastening system.

The brace 30 preferably also includes a plurality of apertures 36 formed therethrough. The apertures 36 preferably are attachment points disposed for accepting fasteners that facilitate attachment of the brace to a foot frame disposed about the foot. For example, as described above in the Background of the Invention section of the application, adjustable rods are well known for providing rigidity between a foot plate arranged proximate a patient's foot and a ring suspended about the lower leg. Instead of the apertures 36, or in conjunction with the apertures, the brace may include other attachment mechanisms comprising attachment points for attachment of the rods. These attachment points may include any known attachment mechanisms, including clips, clamps, and threaded bores, for example.

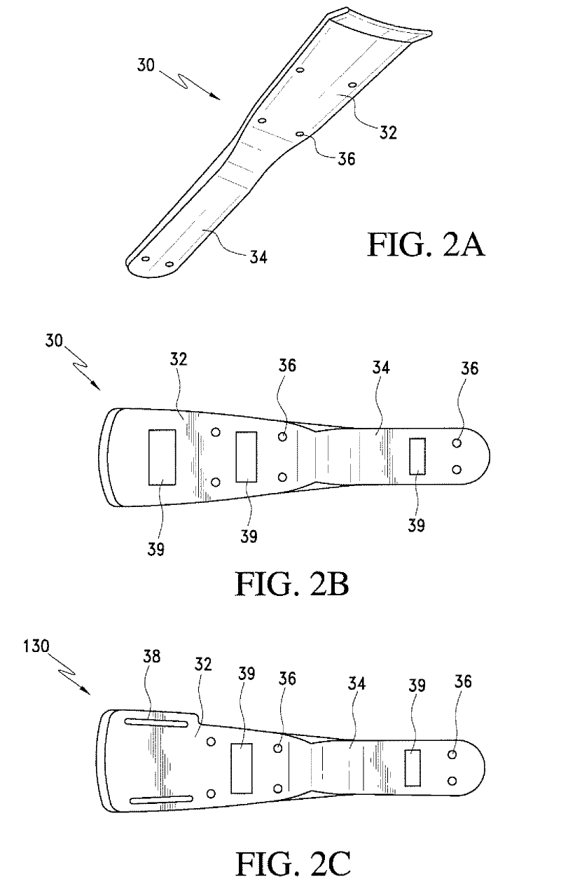

The braces 30 preferably are secured over the sheath 20 using a plurality of straps 40. The straps 40 preferably are adjustable to ensure securement of the braces about the eg. Any known fastening means can be employed to secure the straps, but hook and loop fasteners are used in the preferred embodiment. One or both of the braces can include additional fasteners for attaching the straps 40 to the braces 30. As shown in FIG. 2C for example, at least one slotted aperture 38 can be included in an alternate grace 130. Preferably, while the slotted aperture may be formed in a flange depending from a side of the brace 130, it preferably is formed through the plane of the upper portion 32. In this manner, the straps 40 remain substantially in the same plane as the braces 40, to insure more even load distribution on the leg. One of the straps 40 may be passed through the aperture to facilitate tightening of the braces about the leg. Alternatively (or additionally) the strap may be attached to a side of the aperture. Moreover, tabs or strips 39 of a hook and loop fastening system may be provided on the braces and the straps may be operable with these strips or tabs, Such attachments limit the relative movement of the braces with respect to the strips, to further ensure that the braces are maintained about the leg.

As discussed above, the immobilization device of the present invention is used in conjunction with a deformity or fracture in the foot and/or ankle. More specifically, the deformity or fracture in one or both of the foot and ankle is repaired by passing one or more trans fixation wires through the patient's foot and/or ankle. Ends of the wires are then fixed to a foot frame arranged proximate the foot and/or ankle, as is generally known in the art. The preferred immobilization apparatus described above is then used to substantially prohibit movement of the foot relative the leg. More specifically, once the wires are fixed to the foot plate, the sheath is secured around the patient's lower leg and secured using the fasteners. The braces are then positioned opposite each other, e.g., on left and right sides of the patient's leg. The straps 30 are tightened and fastened to secure the braces in position, with the attachment points exposed when the straps are applied. In this manner, the apertures are accessible for attachment of a first end of an immobilization rod. The second, opposite end of the immobilization rod is fixed to the foot plate.

So applied, the present invention provides a brace for use with a conventional foot frame and immobilization rods. As will be understood, the present invention does not require wires to be placed through either the tibia or the fibula of the patient. Nor does it require bulky braces or boots. Moreover, the apparatus is easily removable from the lower leg, for example, to clean the patient's legs or when articulation of the foot with respect to the leg is required during the recovery process, even before the wires are removed from the foot.

As illustrated in FIG. 3, the invention may further include a foot pad 60 for selective attachment proximate the sole of the patient's foot. As depicted, the foot pad 50 preferably is secured to the foot using a plurality of straps 52. The foot pad has a tread 54 on a bottom thereof, and preferably is cushioned for the comfort of the patient. The foot pad eases the patient's mobility.

Although the present invention has been described with reference to a preferred embodiment, alternative embodiments also are contemplated. For example, it is contemplated that the sheath 20 may not be used. The sheath is generally used to provide a protective layer between the patient's skin and the braces so that the braces do not dig into the patient's leg or otherwise irritate the patient's skin. However, it is understood that the invention may be used without the sheath.

Along the same lines, in a further embodiment of the invention a padded backing or the like may be applied to the side of each of the braces which is to contact the patient's leg. In this embodiment, the sheath likely would not be used in as much as the brace would also provide padding between the patient's leg and the rigid material used to construct the brace.

Although the present invention has been described for use in maintaining a foot immobilized with respect to a lower leg, it is understood that the invention also could be used to immobilize other parts of the body with respect to each other. For example, the apparatus of the present invention may be applied to the forearm, to keep a patient's hand from moving relative to the arm.

The foregoing embodiments of the invention are representative embodiments, and are provided for illustrative purposes. The embodiments are not intended to limit the scope of the invention. Variations and modifications are apparent from a reading of the preceding description and are included within the scope of the invention. The invention is intended to be limited only by the scope of the accompanying claims.

1. An external fixation device for substantially immobilizing a patient's foot relative to the patient's leg, the foot having pins disposed therethrough with opposite ends of the pins being secured to a foot frame arranged proximate the patient's foot, the fixation device comprising:
a pair of braces adapted for arrangement on opposite sides of the patient's leg, each of the braces having a plurality of attachment points for receiving a first end of a stabilizing rod;
a cushion disposed between each of the braces and the patient's leg; and
a fastener for securing the braces and the cushion relative to the patients leg,
wherein the stabilizing rod is adjustably secured proximate a second end to the foot frame.

2. The external fixation device according to claim 1, wherein the attachment points comprise apertures formed through the braces.

3. The external fixation device according to claim 1, wherein the attachment points comprise one of a clip, a clamp, and a threaded bore.

4. The external fixation device according to claim 1, wherein the cushion comprises a sleeve disposed on a portion of the patient's leg.

5. The external fixation device according to claim 1, wherein the cushion comprises two cushions, each of the cushions being attached to one of the braces.

6. The external fixation device according to claim 1, wherein the fastener comprises at least one adjustable strap.

7. An external fixation apparatus for use in immobilizing a foot with respect to an ankle, the apparatus comprising:
a plurality of pins for insertion through a patient's foot;
a foot frame encompassing at least a portion of the patient's foot, the foot frame including a plurality of attachment points for attachment of distal ends of the pins;
a plurality of stabilizing rods having a first end and a second end, the first end being selectively attachable to the foot frame;
a selectively attachable sleeve for placement on the patient's leg;
a pair of braces disposed along a portion of the patient's leg, on the sleeve, each brace having a plurality of apertures therethrough providing attachment points for selective attachment of the second end of the stabilizing rods;
a fastener for maintaining the braces relative to the sleeve; and
a foot plate having a tread formed on a bottom thereof for selective attachment relative to the sole of the patient's foot.

8. The external fixation apparatus according to claim 7, wherein the sleeve is formed of a textile.

9. The external fixation apparatus according to claim 7, wherein the sleeve comprises an adjustor for adjusting the inner diameter of the sleeve to secure the sleeve on the patient's leg.

10. The external fixation apparatus according to claim 9, wherein the adjustor comprises a hook and loop fastener disposed on the sleeve

11. The external fixation apparatus according to claim 10, wherein the sleeve is formed of a fabric comprising the loop portion of the hook and loop fastener, and the hook portion of the hook and loop fastener is adhered to the fabric.

12. The external fixation apparatus according to claim 7, wherein the braces are substantially rigid.

13. The external fixation apparatus according to claim 7, wherein each of the braces is contoured to generally align with contours of the patient's leg.

14. The external fixation apparatus according to claim 7, wherein the fastener comprises at least one adjustable strap.

15. The external fixation apparatus according to claim 7, wherein the fastener comprises a hook and loop fastener.

16. The external fixation apparatus according to claim 7, wherein the foot plate comprises a base having the tread formed on the bottom thereof and at least one adjustable strap for securing the foot plate to at least one of the foot frame, the patient's foot, and the securing rods.

17. An external fixation device for use with a conventional foot immobilization technique, the technique comprising inserting pins through a patient's foot and securing the pins to a foot frame at least partially surrounding the patient's foot, the fixation device comprising
a sleeve, open on opposite ends, selectively attachable over a portion of the patient's leg;
a pair of braces disposed on the sleeve on opposite sides of the patient's leg, generally along the patient's leg; and
at least one removable strap for securing at least one of the sleeve and the braces relative to tile patient's leg,
wherein a plurality of rods are secured proximate opposite ends to the foot frame and one of the braces to substantially inhibit movement of the foot frame relative to the braces, and thus immobilize the patient's foot relative to the patient's leg.

18. The external fixation apparatus according to claim 17, wherein the sleeve comprises a fabric.

19. The external fixation apparatus according to claim 17, the braces further comprising a plurality of attachment facilitators.

20. The external fixation apparatus according to claim 17, further comprising a foot plate having a tread thereon, the foot plate being selectively attachable, proximate the sole of the patient's foot.
